(19) United States
(12) Patent Application Publication
(10) Pub. No.: US 2012/0035357 A1
(43) Pub. Date: Feb. 9, 2012

(54) PROCESS FOR THE PREPARATION OF CARBAPENEM ANTIBIOTIC

(75) Inventors: Sureshkumar Kanagaraj, Tamil Nadu (IN); Senthilkumar Udayampalayam Palanisamy, Tamil Nadu (IN); Maruthi Chandrasekhara Kishor Addanki, Tamil Nadu (IN); Vinod Babu Dasari, Tamil Nadu (IN); John Bosco John Peter, Tamil Nadu (IN); Karthikeyan Lakshmi Narayanan, Tamil Nadu (IN)

(73) Assignee: ORCHID CHEMICALS & PHARMACEUTICALS LTD., Chennai, Tamil Nadu (IN)

(21) Appl. No.: 13/146,161

(22) PCT Filed: Feb. 25, 2010
(86) PCT No.: PCT/IB10/00372

§ 371 Date: Oct. 19, 2011

(30) Foreign Application Priority Data
Feb. 26, 2009 (IN) 424/CHE/2009

(51) Int. Cl. C07D 477/20

(52) U.S. Cl. 540/350

(57) ABSTRACT

The present invention relates to an improved process for the preparation of the carbapenem antibiotic of formula (I) or its salts, hydrates and esters. The present invention further provides novel crystalline form of compound of general formula (III), wherein R³ is p-nitrobenzyloxy carbonyl.

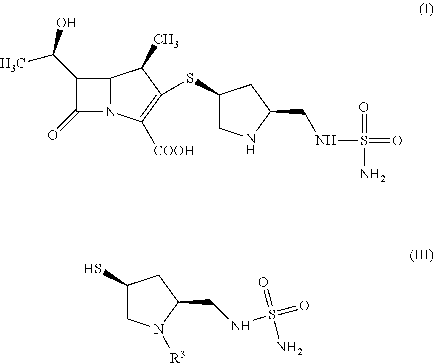

### FIELD OF THE INVENTION

The present invention relates to an improved process for the preparation of the carbapenem antibiotic of formula (I) or its salts, hydrates and esters. Particularly the present invention relates to the preparation of Doripenem of formula (I) or its salts, hydrates and esters.

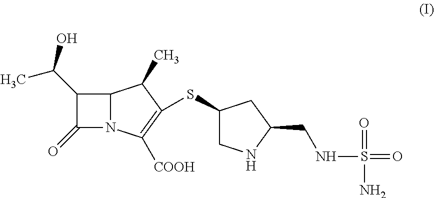

The present invention further provides novel crystalline form of compound of general formula (III), which is an important key raw material in the preparation of Doripenem.

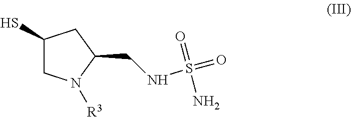

wherein R³ represents p-nitrobenzyloxy carbonyl.

### BACKGROUND OF THE INVENTION

Doripenem, is a synthetic broad-spectrum carbapenem antibiotic structurally related to β-lactam antibiotics. The chemical name for Doripenem is (4R,5S,6S)-3-[((3S,5S)-5-[[(aminosulfonyl)amino]methyl]-3-pyrrolidinyl)thio]-6-[(1R)-1-hydroxyethyl]-4-methyl-7-oxo-1-azabicyclo[3.2.0]hept-2-ene-2-carboxylic acid. Doripenem in the form of monohydrate is marketed in United States of America as DORIBAX™

It is indicated as a single agent for the treatment of complicated urinary tract infections, including pyelonephritis caused by Escherichia coli including cases with concurrent bacteremia, Klebsiella pneumoniae, Proteus mirabilis, Pseudomonas aeruginosa, and Acinetobacter baumannii.

Various methods are reported in the prior art for the preparation of compound of formula (I) which includes the condensation of compound of formula (II) with compound of formula (III) and subsequent deprotection of the protecting group as shown in scheme-1.

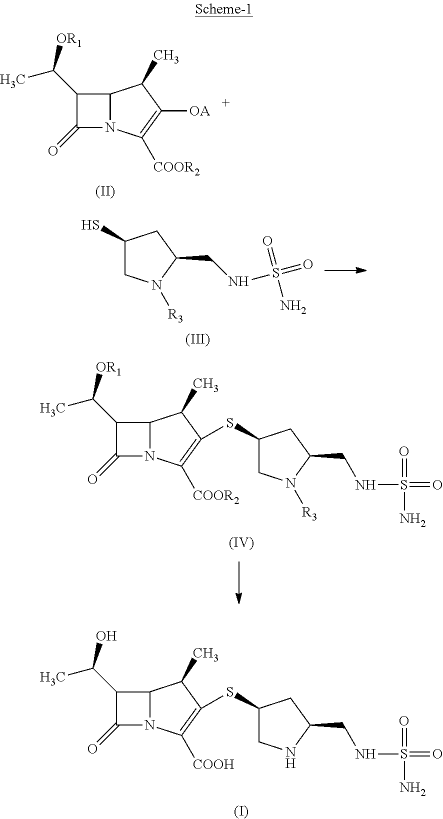

wherein R¹ is hydrogen or hydroxy protecting group such as tert-butyl dimethyl silyl and the like, R² is hydrogen or carboxyl protecting group such as p-nitrobenzyl, p-methoxy benzyl, allyl and the like, R³ is hydrogen or amino protecting group such as p-nitrobenzyloxy carbonyl, allyloxy carbonyl and the like and A is an activating group such as P(O)(OR)₂, SO₂R and the like wherein R is selected from substituted or unsubstituted C₁₋₆ alkyl, aralkyl or aryl to form the compound of formula (II).

Doripenem is first disclosed in U.S. Pat. No. 5,317,016, which also discloses a process for the preparation of same. This patent discloses various methods for the preparation of compound of general formula (III) followed by condensation with compound of general formula (II) using base such as N-ethyldiisopropylamine and subsequent deprotection yields Doripenem, isolated by lyophilization technique. Further this patent utilizes column chromatographic technique for the isolation of compound of general formula (III).

U.S. Pat. No. 5,872,250 discloses a process for the preparation of Meropenem and Ertapenem, which involves the use of secondary amine base selected from diisopropylamine (DIPA), dicyclohexylamine (DCHA), 2,2,6,6-tetramethylpiperidine (TMP), 1,1,3,3-tetramethylguanidine (TMG), 1,8-diazabicyclo[4.3.0.]undec-7-ene (DBU) and 1,5-diazabicyclo[4.3.0]non-5-ene (DBN) for the condensation of enol phosphate with corresponding mercapto compound.

In general the β-lactam antibiotics are sensitive towards both acids and bases and hence during condensation of compound of general formula (III) with compound of general formula (II) in the presence of base such as either secondary amine or tertiary amine tend to degrade the product thereby producing the compound of formula (I) which contain unwanted impurities. The use of base such as secondary or tertiary amine for the condensation reaction resulted in extended reaction time thereby not viable in large scale preparation.

U.S. Pat. No. 6,111,098 disclose two types of crystal namely type-1 & type-2 and process for the preparation of same. This patent also utilizes lyophilization technique for the isolation of said crystalline Doripenem.

US 2003/0153191 disclose another two types of crystal namely type-3 & type-4 and process for the preparation of same. The process involves isolation of said crystalline Doripenem hydrate by crystallization technique.

JP 2003/026680 disclose process for the preparation of Doripenem without isolating the mercapto compound of general formula (III) by condensing with compound of general formula (II) thereby isolating the Doripenem in lower yield which also contains unwanted impurities. This further requires additional purification to remove the unwanted impurities thereby increasing the cost of production which in turn not viable in large scale production.

JP 2008/120830 discloses crystalline compound of general formula (IV)

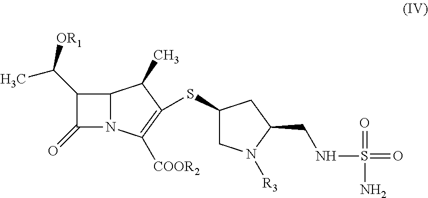

wherein R¹ is hydrogen, R² is p-nitrobenzyl, R³ is p-nitrobenzyloxycarbonyl. Further this publication discloses a process which involves the use of ethyl acetate solution of compound of formula (III) without isolation for condensation with compound of formula (II). In such a case the exact quantity of raw material can not be measured and hence either one of the raw materials will be present in the reaction mixture or chance for the formation of impurities is high leading to the production of less pure Doripenem.

IN 1104/DEL/2005 and its equivalent WO 2006/117763 discloses a process for the preparation of Doripenem by condensing compound of formula (II) and (III) to produce compound of formula (IV) followed by deprotection with out isolating the compound of formula (IV). In such a case the impurities formed during condensation reaction is carried forward in the deprotection stage leading to production of less pure Doripenem. This patent also discloses the isolation of amorphous Doripenem by anti solvent precipitation.

IN 2370/DEL/2005 and its equivalent WO 2007/029084 discloses the use of acetyl chloride, silica chloride, thionyl chloride or oxalyl chloride for the preparation of mercapto compound of general formula (III) from compound of general formula (V). Further this patent does not isolate compound of general formula (III) and used as an oily residue for further reaction.

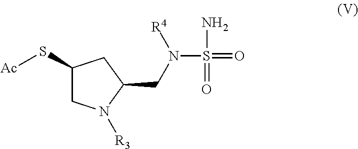

wherein R³ and R⁴ are same or different and selected from is hydrogen or amino protecting group. Since the said application uses oily residue with out purification, the impurities and by-products formed during the reaction carried forward to the next stage leading to the production of less pure Doripenem in lower yield. This patent utilizes acetyl chloride for the deacetylation to prepare the compound of formula (III). Use of catalytic amount of acetyl chloride leads to lesser formation of product. Moreover, the reaction didn't proceed further whereas the present invention utilizes Phosphorous oxychloride for the deacetylation. Use of Phosphorous oxychloride even in catalytic amount gave good product formation.

All the prior art reported utilizes compound of formula (III) as a pasty mass OR for isolation of compound of formula (III) column purification is required. None of the prior arts provides the simple isolation of compound of formula (III) as crystalline form even as a solid form.

With our continued research for developing a process for the preparation of compound of formula (I) or its salts, hydrates and esters, we have identified a process, in which the compound of general formula (II) is condensed with compound of general formula (III) in the presence of anionic exchange resin and subsequent deprotection followed by isolating the product by simple precipitation technique with high purity thereby producing Doripenem in good yield with high purity. None of the prior art suggested or even motivated the present invention and hence this constitutes the novelty of the present invention.

### OBJECTIVES OF THE INVENTION

The main objective of the present invention is to provide a simple and commercially viable, industrially scalable process for the preparation of compound of the formula (I) or its salts, hydrates and esters.

Another objective of the present invention is to provide a simple and commercially viable, industrially scalable process for the isolation of compound of general formula (III), which avoids chromatographic techniques.

Yet another objective of the present invention is to provide a simple and commercially viable, industrially scalable process for the preparation of compound of general formula (IV).

### SUMMARY OF THE INVENTION

Accordingly, the primary aspect of the present invention is to provide an improved process for preparation of compound of the formula (I) or its salts, hydrates and esters.

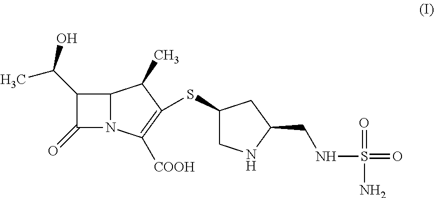

the said process comprising the steps of
i) treating compound of general formula (II)

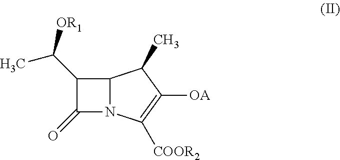

wherein R¹ is hydrogen or hydroxy protecting group, R² is hydrogen or carboxyl protecting group and A is an activating group such as P(O)(OR)₂, SO₂R and the like and R is selected from phenyl or methyl with compound of general formula (III)

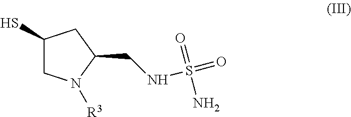

wherein R³ is p-nitrobenzyloxy carbonyl in the presence of anion exchange resin optionally in the presence of solvent;
ii) quenching the step (i) reaction mass in solvent(s);
iii) optionally isolating the compound of formula (IV);

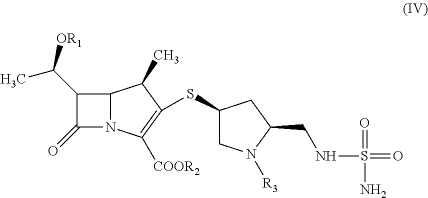

wherein R¹ is hydrogen or hydroxy protecting group, R² is hydrogen or carboxy protecting group, R³ is p-nitrobenzyloxy carbonyl.
iv) deprotecting the protecting group in a solvent, hydrogen pressure and in the presence of metal catalyst;
v) extracting Doripenem in water; and
vi) isolating the doripenem compound of formula (I) or its salts, hydrates and esters optionally by the addition of first antisolvent followed by second antisolvent.

In the second aspect of the present invention, it provides an improved process for the isolation of Doripenem in amorphous form, the said process comprising the steps of:

1) obtaining a solution of Doripenem in water;

2) adding first anti solvent;

3) adding second anti solvent to precipitate; and

4) isolating Doripenem in amorphous form

In third aspect of the present invention, it provides process for the isolation of compound of formula (III), the said process comprising the steps of

a) optionally deacylating the compound of formula (V)

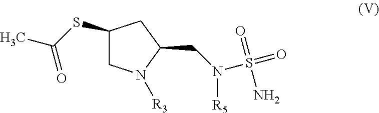

wherein R³ is p-nitrobenzyloxy carbonyl, R⁵ represents hydrogen or tert-butoxycarbonyl(t-Boc)

b) obtaining solution of compound of formula (III) in solvent(s);

c) optionally removing the solvent(s);

d) mixing with anti solvent; and

e) isolating compound of formula (III).

In fourth aspect of the present invention, it further provides novel crystalline compound of formula (III)

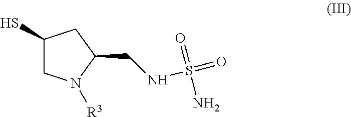

wherein R³ is p-nitrobenzyloxy carbonyl, having substantially the same X-ray diffractogram as set out in FIG. 1.

### DESCRIPTION OF THE FIGURES

FIG. 1: Powder XRD pattern of novel crystalline form of compound of formula (III);

FIG. 2: Powder XRD pattern of amorphous form of compound of formula (I) Doripenem; analyzed by X-Ray Powder Diffractometer of following features:

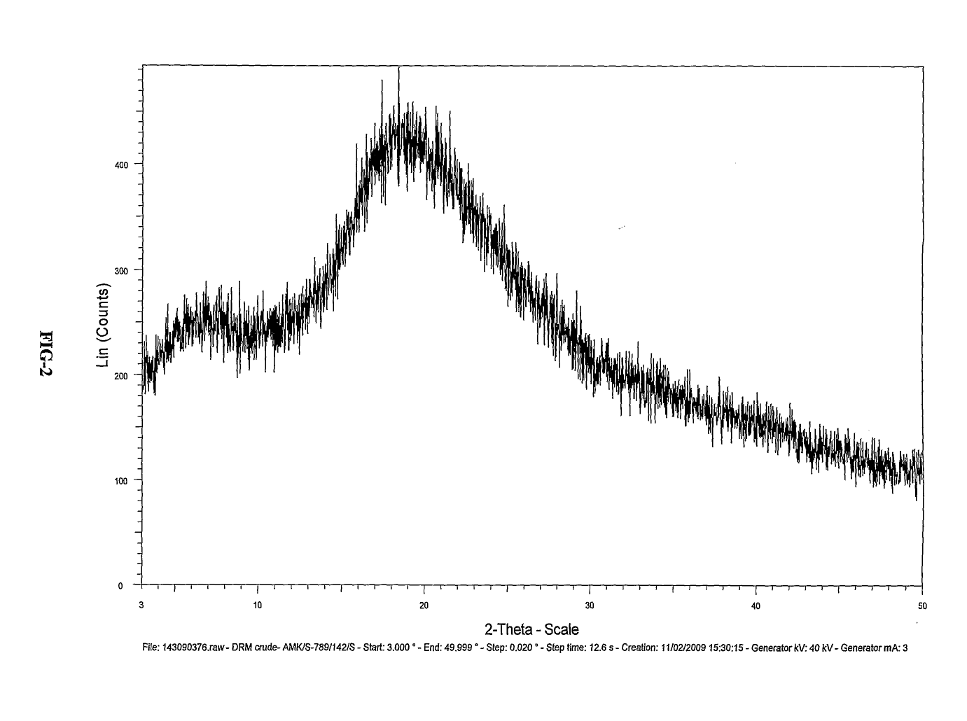

| | |
|---|---|
| Make | BRUKERAXS |
| Model | D8 ADVANCE |
| Data handling system | EVA 12.0.0.0. |
| ANODE | COPPER |
| RADIATION | COPPER K alpha-1 |
| WAVELENGTH | 1.5406 A° |
| CURRENT &VOLTAGE | 40 kV 30 mA |

### DETAILED DESCRIPTION

In an embodiment of the present invention hydroxy protecting group represented by R¹ is selected from the group comprising trimethylsilyl, triethylsilyl, t-butyldimethylsilyl, triisopropylsilyl, triphenylmethyl and the like, preferably t-butyldimethylsilyl.

In an another embodiment of the present invention carboxyl protecting group represented by R² is selected from the group comprising p-nitrobenzyl, p-methoxy benzyl, allyl, vinyl, tertiary butoxycarbonyloxymethyl, methoxycarbonyloxymethyl and the like, preferably p-nitrobenzyl.

In an embodiment of the present invention, the anion exchange resin used in step (i) is selected from Amberlite LA-2, Amberlite IRA-67, Amberlite IRA-68, Amberlite IRA-94 S, Amberlite IRA-92, Amberlite IRA-96, and the like or mixtures thereof. As the ion exchange resins mentioned were only identified by their trade name Details about the nature of resin can be obtained in: http://www.chemicalbook.com/ProductChemicalPropertiesCB4116653_EN.htm

Secondary-amine-type ion-exchange resins which are suitable for the process of this invention are liquid and have a high molecular weight. Such resins are oil soluble, but water insoluble. Preferably, the resin will have a molecular weight in the range of 350-400 and a capacity of approximately 2.5-2.8 meq/g (2.1-2.3 meq/mL). Examples of resins which can be used are Amberlites LA-1 and LA-2 (Rohm & Haas, Philadelphia, Pa. 19105), Amberlite IRA-92. The use of anionic exchange resin for the condensation reaction is helpful in yielding the final compound with high purity. Further the reaction proceeds smoothly as well as the rate of reaction is faster. In addition to this the resin can be easily removed from the reaction mixture by simple filtration and can be reused after proper activation of the resin, which constitutes one of the novelty of the present invention.

In another embodiment of the present invention, the solvent used in step (i) is selected form acetonitrile, acetone, ethyl acetate, N,N-dimethylformamide (DMF), N,N-dimethylacetamide (DMAc), N-methylpyrrolidone (NMP), sulfolane monoglyme, diglyme and the like or mixtures thereof.

In still another embodiment of the present invention, the solvent used in step (ii) for the quenching of reaction mass is selected from the group comprising water, diisopropyl ether, toluene, heptane, cyclohexane, hexane and the like or mixtures thereof. The solvent may optionally contain the buffer selected from potassium dihydrogen orthophosphate, dipotassium hydrogen orthophosphate, MOPS, sodium dihydrogen orthophosphate, orthophosphoric acid, sodium sulphate, EDTA, and the like or mixtures thereof. Preferably the pH of the water solution is adjusted to 6.0 to 8.0.

The compound of general formula (IV) either can be isolated by conventional technique or can be used with out isolation for deprotection of the protecting groups.

In yet another embodiment of the present invention, the solvent used in step (iii) for the deprotection of compound of formula (IV) is selected from the group comprising tetrahydrofuran, ethyl acetate, dichloromethane, methanol, ethanol acetonitrile, water and the like or mixtures thereof optionally in the presence of base selected form sodium bicarbonate, sodium carbonate, sodium hydroxide and the like or mixtures thereof by using metal catalysts such as palladium carbon, raney nickel, platinum carbon, Pd/C in presence of calcium carbonate and in the presence of hydrogen.

In an embodiment of the present invention, the compound of formula (I) is isolated by conventional technique such as lyophilization, crystallization and the like.

In one more embodiment of the present invention, it provides a simple isolation technique to isolate the compound of formula (I) in amorphous form. Accordingly to a solution of doripenem in water two anti solvents were added one followed by another, this mode of addition always yield Doripenem or its hydrate in an amorphous form with good purity having good flow property, which is an essential physical property for an active pharmaceutical ingredients. Applicant further observed that the use of single anti solvent addition leads to formation of Doripenem as pasty mass. A solution of Doripenem in water is obtained either by dissolving doripenem in water or directly from the reaction.

Accordingly the first anti solvent used in step (vi) and step (2) for the isolation of Doripenem in amorphous form is selected from acetone, methyl ethyl ketone, methyl isobutyl ketone, diethyl ketone, tetrahydrofuran and the like or mixtures thereof.

In still another embodiment of the present invention, the second anti solvent used in step (vi) and step (3) for the isolation of Doripenem in amorphous form is selected from methanol, ethanol, isopropyl alcohol n-butanol, t-butanol, isobutanol, acetonitrile and the like or mixtures thereof.

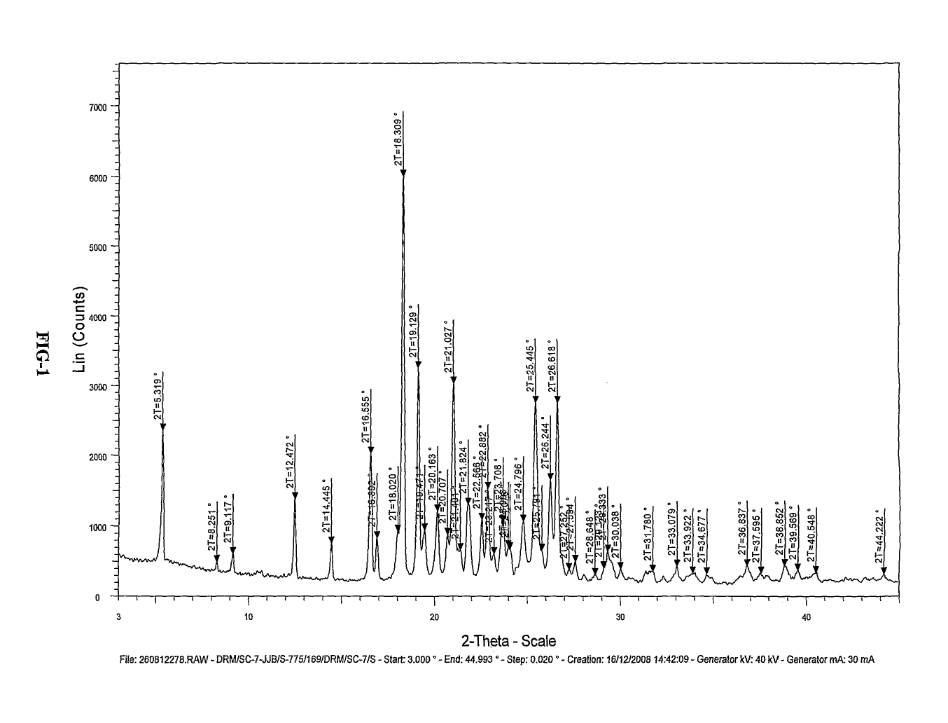

In an embodiment of the present invention, it further provides novel crystalline compound of formula (III)

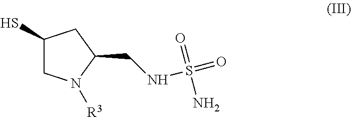

wherein R3 is p-nitrobenzyloxy carbonyl, having substantially the same X-ray diffractogram as set out in FIG. 1.

All the reported prior arts yields the compound of formula (III) only as an oily residue form which contains impurities and the said form is inconvenient to store, whereas the present invention provides compound of formula (III) as a crystalline form which possess good stability and high purity. It would be desirable to form such a crystalline form as the crystalline form has enhanced storage stability, good ease of handle. The achieved high purity of compound of formula (III) makes the final compound higher purity than the compound obtained by the other methods.

In another embodiment of the present invention the reagents employed in step (a) for deacetylation of compound of formula (V) is selected from Phosphorous oxychloride, thionyl chloride, sulphuric acid, sodium methoxide, sodium hydroxide, Diaion Lewatit® K 2649, UBK 530, Diaion UBK 535, Diaion UBK 550, Diaion UBK 555, Diaion SK 104, Diaion SK 1B, Diaion SIAL 10, Diaion PK 208, Diaion PK 220, Relite RPS, Relite CFZ, Relite CND and the like or mixtures thereof, preferably Phosphorous oxychloride and Lewatit® K 2649. Lewatit® K 2649 is a strongly acidic, macroporous, polymer-based catalysts in spherical bead form, with sulfonic acid groups. Details can be viewed at http://www.sybronchemicals.net/products/organic/k2649.pdf. Use of phosphorous oxychloride even in catalytic amount gave good product formation.

In still another embodiment of the present invention, a solution of compound of formula (III) in solvent(s) is obtained either by dissolving the compound of formula (III) in suitable solvent or directly from the reaction mixture. According a solution of compound of formula (III) is obtained by using the technique available in the prior art or by following the procedure given in the reference examples.

In yet another embodiment of the present invention, the solvent used in step (b) for the isolation of compound of general formula (III) is selected from the group comprising dichloromethane, ethyl acetate, methanol, ethanol, isopropyl alcohol, chloroform and the like or mixtures thereof.

In another embodiment of the present invention, the solvent used in step (b) is optionally removed by conventional technique like distillation, evaporation and the like.

In one more embodiment of the present invention, the anti solvent used in step (d) for the isolation of compound of general formula (III) is selected from the group comprising toluene, diisopropyl ether, cyclohexane, heptane, hexane, pentane and the like or mixtures thereof.

In still another embodiment of the present invention, the compound of general formula (III) is isolated preferably in crystalline form.

Applicant found that the use of resin for the deacetylation proceeds smoothly and results in good yield. Accordingly the present invention provides a process for the preparation of compound of general formula (Y), which comprising the steps of:

A) deacetylating the compound of general formula (X) using phosphorus oxychloride or a resin,

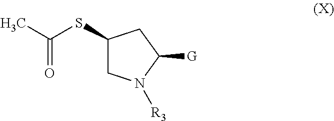

B) isolating compound of general formula (Y)

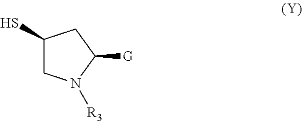

wherein G represents hydrogen or a group of formula,

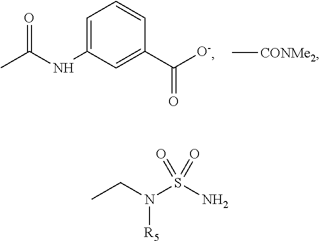

(wherein R5 is hydrogen or tert-butoxycarbonyl), and R³ represents p-nitrobenzyloxycarbonyl OR a chemical formula

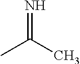

In another embodiment of the present invention, the resin employed in the step A is selected from the group consisting Lewatit® K 2649, Diaion UBK 530, Diaion UBK 535, Diaion UBK 550, Diaion UBK 555, Diaion SK 104, Diaion SK 1B, Diaion SKL 10, Diaion PK 208, Diaion PK 220, Relite RPS, Relite CFZ, Relite CND and the like, preferably Lewatit® K 2649.

Commercial availability of the above ion-exchange resins, their complete and easy removal from the reaction medium by simple filtration and their regeneration make this process commercially economical. Diaion UBK 530, Lewatit® K 2649 are more preferred because of their separation efficiency is high.

As the ion exchange resins mentioned were only identified by their trade name Details about the nature of resin can be obtained in http://www.diaion.com/Diaion_Tables/Diaion_CationTable_R_E.htm

In one more embodiment of the present invention the starting material of the present invention namely compound of formula (II) and (III) can be prepared by conventional method or by following the procedure provided in the reference examples.

The following examples are provided by way of illustration only and should not be construed to limit the scope of the invention.

### Preparation of Protected Doripenem

### Example 1-3

### Example 1

To a solution of Enol phosphate of formula (II) (1.4 g) in DMF (10 mL) was added (2S,4S)-1-p-nitrobenzyloxycarbonyl-2-sulfamoylaminomethyl-4-mercaptopyrrolidine (1.1 g) and cooled to −25° C. A solution of Amberlite LA-2 resin (1.4 g) in ethyl acetate (8 ml) was added. The reaction mixture was stirred at −25 to −30° C. After completion of reaction, a mixture ethyl acetate (25 mL) and purified water (25 mL) was added to the reaction mixture. The organic layer was separated and the aqueous layer was extracted with ethyl acetate. The combined organic layer was successively washed with purified water, dil. hydrochloric acid and saturated sodium chloride solution. The solvent was removed by distillation and added diisopropyl ether. The solid formed was filtered, washed with diisopropyl ether and drying afforded pure amorphous product.

### Example-2

To a solution of Enol phosphate of formula (II) (1.4 g) in DMF (10 mL) was added (2S,4S)-1-p-nitrobenzyloxycarbonyl-2-sulfamoylaminomethyl-4-mercaptopyrrolidine (1.1 g) and cooled to −25° C. Amberlite LA-2 resin (1.4 g) was added and stirred at −25 to −30° C. After completion of reaction, the reaction mixture was added into purified water containing dipotassium hydrogen orthophosphate, sodium sulphite and EDTA whose pH is adjusted to neutral with orthophosphoric acid. The pH was then adjusted to 4.0 using dil. sulfuric acid. The product formed was filtered, washed and suck dried afforded pure title compound.

### Example 3

To a solution of Enol phosphate (105.2 g) in DMF (526 ml) was added (2S,4S)-1-p-nitrobenzyloxycarbonyl-2-sulfamoylaminomethyl-4-mercaptopyrrolidine (70 g) and cooled to −30° C. N-Ethyldiisopropyl amine (26.4 g) in DMF was added slowly at −25 to −30° C. The reaction mixture was stirred. To the reaction mixture ethyl acetate and purified water were added. Organic layer was separated and the aqueous layer was extracted with ethyl acetate. Combined organic layer was washed with purified water. To the clear organic layer, diisopropyl ether was added. The solid formed was stirred and filtered. The product obtained was washed with diisopropyl ether and dried under vacuum to yield the pure amorphous product.

Yield: 121 g

### Advantages of Resin:

Amberlite resin are reusable, thereby overall cost is minimized.

### Preparation of Doripenem

### Example 4-7

### Example-4

To a solution of Doripenem p-nitrobenzyl ester of formula (IV) (where R² is p-nitrobenzyl, R³ is p-nitrobenzyloxycarbonyl) in a mixture of purified water (250 mL) and tetrahydrofuran (452 mL) was added 10% Pd/C (34 gm on dried basis). The resultant mass was hydrogenated till completion of reaction. The carbon was removed by filtration and washed with a mixture of THF and water. The filtrate was washed with ethyl acetate and subjected to carbon treatment in the presence of EDTA, sodium dithionite, nitrogen and vacuum. To the aqueous solution was added acetone (480 mL) followed by isopropyl alcohol (2800 mL). The precipitated product was filtered and washed with acetone. Drying the product under vacuum afforded amorphous Doripenem.

### Advantages:

Addition of acetone or IPA alone to the reaction mass resulted in pasty mass, and does not yield free flowing amorphous powder, where as the use of two anti-solvent one followed by another yielded free flowing amorphous powder.

### Example-5

To a solution of Doripenem p-nitrobenzyl ester (65 g) in mixture of purified water and tetrahydrofuran, Pd/C was charged. After the hydrogenation, reaction mixture was filtered through hyflo bed and washed with water. The filtrate was washed with ethyl acetate. The aqueous layer was separated and charged activated carbon. The resultant mass was stirred and filtered. To the filtrate acetone (1300 ml) was charged followed by isopropyl alcohol (6500 ml). The resultant mass was stirred. The solid formed was filtered and washed with acetone. The product obtained was dried under vacuum to yield the amorphous doripenem (24.7 g, HPLC purity 98.6%).

### Example-6

To a solution of (2S,4S)-1-p-nitrobenzyloxycarbonyl-2-sulfamoylaminomethyl-4-mercaptopyrrolidine in DMF were added Enol phosphate of formula II (36.5 g) and Amberlite LA-2 resin at −10 to −15° C. After completion of reaction, was added into purified water containing dipotassium hydrogen orthophosphate, sodium sulphate, and EDTA whose pH was adjusted to neutral by using orthophosphoric acid. The reaction mixture pH was again adjusted to 3.0-5.0 using dil. sulfuric acid. The product formed was filtered, washed with water and suck dried. Without further drying this material is used for deprotection.

To a solution of Doripenem p-nitrobenzyl ester in a mixture of purified water (226 mL) and tetrahydrofuran (450 mL) was added 10% Pd/C (43 g, on dried basis). The resultant mass was subjected to hydrogenation till completion of reaction. Carbon was removed by filtration and the filtrate was washed with ethyl acetate. The aqueous layer was treated with activated carbon-ENO in the presence of nitrogen and vacuum and optionally in the presence of EDTA and sodium dithionite. To the aqueous layer were added acetone (460 mL) and ethanol (2800 mL). The precipitated product was filtered and washed with acetone. Drying the product under vacuum afforded amorphous Doripenem.

### Example-7

To a solution of (2S,4S)-1-p-nitrobenzyloxycarbonyl-2-sulfamoylaminomethyl-4-mercaptopyrrolidine in DMF were added Enol phosphate of formula II and Amberlite LA-2 at −10 to −15° C. and maintained till completion of reaction. The reaction mixture was added into mixture of THF and purified water containing dipotassium hydrogen orthophosphate, sodium sulphate, and EDTA whose pH was adjusted to neutral by using orthophosphoric acid. The reaction mixture pH was again adjusted to 3.0-5.0 using dil. sulfuric acid. To the separated THF layer was added purified water and 10% Pd/C. The resultant mass was subjected to hydrogenation till completion of reaction. Carbon was removed by filtration and the filtrate was washed with ethyl acetate. The aqueous layer was treated with activated carbon-ENO in the presence of EDTA, sodium dithionite, nitrogen and vacuum. To the aqueous layer were added acetone and methanol. The precipitated product was filtered and washed with acetone. Drying the product under vacuum afforded amorphous Doripenem.

### Preparation of (2S,4S)-1-p-nitrobenzyloxycarbonyl-2-sulfamoylaminomethyl-4-mercaptopyrrolidine compound of formula (III)

### Example a-f

### Example-a

To a slurry of (2S,4S)-4-Acetylthio-2-(N-sulfamoyl-tert-butoxycarbonylaminomethyl)-1-(4-nitrobenzyloxycarbonyl)pyrrolidine (5 g) in methanol (30 mL) was added phosphorous oxychloride (2 g). The resultant mixture was heated to reflux till completion of reaction. To the cooled reaction mixture were added ethyl acetate (25 mL), purified water (10 mL) and saturated sodium chloride solution (15 mL) were added and separated the organic layer. The aqueous layer was extracted with ethyl acetate and the combined organic layer was washed successively with purified water, saturated sodium bicarbonate solution and saturated sodium chloride solution. Solvent was removed and to the residue was added toluene (20 mL). The solid formed was filtered and washed with diisopropyl ether. Drying the product afforded pure crystalline title compound.

### Example-b

To a slurry of (2S,4S)-4-Acetylthio-2-(N-sulfamoyl-tert-butoxycarbonylaminomethyl)-1-(4-nitrobenzyloxycarbonyl)pyrrolidine (10 g) in methanol (50 mL) was added sulfuric acid (4.7 g) and the resultant mixture was heated to 65-70° C. and maintained till completion of reaction. To the cooled reaction mixture were added ethyl acetate (100 mL), purified water (80 mL) and saturated sodium chloride solution (50 mL) and separated the organic layer. The organic layer was added into diisopropyl ether. The solid formed was filtered and washed with diisopropyl ether. Drying the product under vacuum afforded pure title compound.

### Example-c

To a slurry of (2S,4S)-4-Acetylthio-2-(N-sulfamoyl-tert-butoxycarbonylaminomethyl)-1-(4-nitrobenzyloxycarbonyl)pyrrolidine (2 g) in methanol (20 mL) was added Diaion UBK-530 resin (10 g) and heated to 50-55° C. and maintained till completion of reaction. The resin was removed by filtration and the filtrate was concentrated to thick mass. To the residue was added diisopropyl ether. The solid obtained was filtered and washed with diisopropyl ether. Drying the product under vacuum afforded pure title compound.

### Example-d

To a slurry of (2S,4S)-4-Acetylthio-2-(N-sulfamoyl-tert-butoxycarbonylaminomethyl)-1-(4-nitrobenzyloxycarbonyl)pyrrolidine (1 g) in methanol (5 mL) was added trimethylchlorosilane and heated to 60-65° C. and maintained till completion of reaction. To the cooled reaction mixture were added dichloromethane (50 mL), purified water (10 mL) and saturated sodium chloride solution (15 mL). The organic layer was successively washed with purified water, saturated sodium bicarbonate solution and saturated sodium chloride solution. To the organic layer was added diisopropyl ether. The crystallized product was filtered and washed with diisopropyl ether. Drying the product under vacuum afforded pure title compound.

### Example-e

To a slurry of (2S,4S)-4-Acetylthio-2-(N-sulfamoyl-tert-butoxycarbonylaminomethyl)-1-(4-nitrobenzyloxycarbonyl)pyrrolidine (5 g) in methanol, Lewatit® K 2649—ion exchange resin (5 g) was added at 25-30° C. The resultant mixture was heated to 60-67° C., then cooled to 25-30° C. The reaction mixture was filtered and washed with methanol. The clear filtrate was concentrated to thick mass and dissolved in tetrahydrofuran. The tetrahydrofuran layer was charged slowly into purified water containing diisopropyl ether (15 ml) and the resultant mass was stirred. The solid product formed was filtered and washed with purified water. Drying the product under vacuum afforded pure title compound (3.2 g, HPLC purity 98%).

### Advantages of Resin:

Amberlite resin are reusable, thereby overall cost is minimized.

### Example-f

To a slurry of (2S,4S)-4-Acetylthio-2-(N-sulfamoyl-tert-butoxycarbonylaminomethyl)-1-(4-nitrobenzyloxycarbonyl)pyrrolidine (100 g) in methanol, Phosphorous oxy chloride (7.8 g) was added. The resultant mixture was heated to 63-67° C. then cooled to 25-30° C. To the reaction mixture ethyl acetate, purified water and saturated sodium chloride solution were added and the organic layer was separated. The aqueous layer was extracted with ethyl acetate and the organic layers were combined. Organic layer was concentrated to thick mass and dissolved in tetrahydrofuran. The tetrahydrofuran layer was charged slowly into purified water containing diisopropyl ether and stirred. The solid product formed was filtered and washed with purified water. Drying the product under vacuum afforded pure title compound (61 g, HPLC purity 98.5%).

### Preparation of (2S,4S)-1-p-nitrobenzyloxycarbonyl-2-dimethylaminocarbonyl-4-mercaptopyrrolidine

### Example-i

To a slurry of (2S,4S)-4-Acetylthio-2-(dimethylaminocarbonyl)-1-(4-nitrobenzyloxycarbonyl)pyrrolidine (10 g) in methanol (100 ml), Lewatit® K 2649—ion exchange resin was added at 25-30° C. The resultant mixture was heated to 60-67° C. then cooled to 25-30° C. The reaction mixture was filtered and washed with methanol. To the reaction mixture ethyl acetate, purified water and saturated sodium chloride solution were added and the organic layer was separated. The aqueous layer was extracted with ethyl acetate and the organic layers were combined. Organic layer was concentrated to thick mass and the product was crystallized using toluene. Yield: 6.3 g, HPLC purity 96%

### Example-ii

To a slurry of (2S,4S)-4-Acetylthio-2-(dimethylaminocarbonyl)-1-(4-nitrobenzyloxycarbonyl)pyrrolidine (10 g) in methanol, phosphorous oxychloride (0.96 g) was added at 25-30° C. The resultant mixture was heated to 60-67° C. and then cooled to 25-30° C. To the reaction mixture ethyl acetate, purified water and saturated sodium chloride solution were added and the organic layer was separated. The aqueous layer was extracted with ethyl acetate and the organic layers were combined. Organic layer was concentrated to thick mass and the product was crystallized using n-hexane. Yield: 6.4 g, HPLC purity 95%

### Preparation of 3-(S)-mercapto-1-p-nitrobenzyloxycarbonylpyrrolidine

### Example-iii

To slurry of 3-(S)-Acetylthio-1-p-nitrobenzyloxycarbonylpyrrolidine (5 g) in methanol, Lewatit® K 2649—ion exchange resin (7.5 g) was added at 25-30° C. The resultant mixture was heated to 60-67° C. then cooled to 25-30° C. The reaction mixture was filtered and washed with methanol. The clear filtrate was concentrated to thick mass and dissolved in tetrahydrofuran. The obtained solution was charged into water-Diisopropyl ether mixture and stirred. The product formed was filtered and washed with water. Drying the product under vacuum gave title compound. (4.2 g, HPLC purity: 94%)

### Example-iv

To a slurry of 3-(S)-Acetylthio-1-p-nitrobenzyloxycarbonylpyrrolidine (5 g) in methanol, phosphorous oxychloride (0.4 g) was added at 25-30° C. The resultant mixture was heated to 60-67° C. and then cooled to 25-30° C. To the reaction mixture ethyl acetate, purified water and saturated sodium chloride solution were added and the organic layer was separated. The aqueous layer was extracted with ethyl acetate and the organic layers were combined. Organic layer was concentrated to thick mass and dissolved in tetrahydrofuran. The obtained solution was charged into water-Diisopropyl ether mixture and stirred. The product formed was filtered and washed with water. Drying the product under vacuum gave title compound. (3.6 g, HPLC purity 96%)

### Reference Example-1

### Preparation of Protected Doripenem

To a solution of (2S,4S)-1-p-nitrobenzyloxycarbonyl-2-sulfamoylaminomethyl-4-mercaptopyrrolidine in DMF were added Enol phosphate of formula (III) (36.5 g) and N-ethyldiisopropylamine (8 g) at −10 to −15° C. and maintained till completion of reaction. The reaction mixture was added into purified water containing dipotassium hydrogen orthophosphate, sodium sulphite and EDTA whose pH was adjusted to neutral by using orthophosphoric acid. The pH was again adjusted to 3.0-5.0 using dil. sulfuric acid. The product formed was filtered, washed with water and suck dried. The protected doripenem thus obtained can be used for next stage with or without drying.

### Reference Example-2

### Preparation of Doripenem Monohydrate

To the solution of Doripenem p-nitrobenzyl ester (100 g) in mixture of purified water and tetrahydrofuran, Pd/C was charged. The resultant mass was hydrogenated for 2-4 hours. The reaction mixture was filtered through hyflo bed and washed with water. The filtrate was washed with ethyl acetate. The aqueous layer was given carbon treatment. Then filtered the mass and washed the bed with purified water. To filtrate doripenem monohydrate (0.1 g) was added and cooled and isopropyl alcohol was added. The resultant mass was stirred at 0-5° C. The solid formed was filtered and washed with aqueous isopropyl alcohol, dried under vacuum at 40-50° C. to yield the doripenem monohydrate (33.6 g).

### Reference Example-3

### Preparation of Doripenem Monohydrate from Amorphous Doripenem

To a solution of amorphous doripenem (60 g) in water activated carbon was added and stirred. The reaction mass was filtered and the bed was washed with water. To the clear filtrate, doripenem monohydrate (0.1 g) was added followed by IPA at 0-5° C. The solid formed was filtered and washed with aqueous isopropyl alcohol and dried under vacuum at 40-50° C. to yield the doripenem monohydrate (35.8 g, HPLC purity 99.90%).

1-15. (canceled)

16. A crystalline form of compound of formula (III) 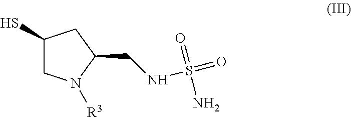
wherein R³ is p-nitrobenzyloxy carbonyl.

17. A process for the preparation of compound of formula (III) as claimed in claim 16, the process comprising the steps of
a) optionally deacetylating the compound of formula (V) 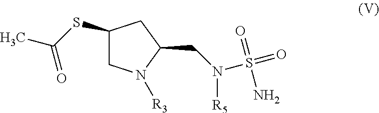
wherein R³ is p-nitrobenzyloxy carbonyl, R⁵ represents hydrogen or tert-butoxycarbonyl(t-Boc)
b) obtaining solution of compound of formula (III) in solvent(s);
c) optionally removing the solvent(s);
d) mixing with anti solvent; and
e) isolating compound of formula (III).

18. A process as claimed in claim 17, wherein the solvent used in step (b) is selected from dichloromethane, ethyl acetate, methanol, ethanol, isopropyl alcohol, chloroform or mixtures thereof.

19. A process as claimed in claim 17, wherein the anti solvent used in step (d) is selected from toluene, diisopropyl ether, cyclohexane, heptane, hexane, pentane or mixtures thereof.

20. The compound of formula (III) as claimed in claim 16, having substantially the same X-ray diffractogram as set out in FIG. 1.

21. The use of compound of formula (III) as claimed in claim 16, in the preparation of Doripenem.

22. An improved process for preparation of compound of the formula (I) or its salts, hydrates and esters, 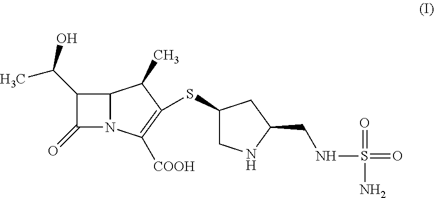
the said process comprising the steps of:
i) treating compound of general formula (II) 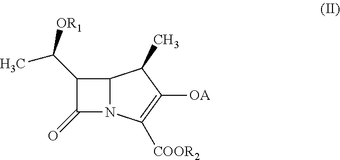
wherein R¹ is hydrogen or hydroxy protecting group, R² is hydrogen or R² is carboxy protecting group, and A is an activating group selected from P(O)(OR)₂, SO₂R wherein R is selected from phenyl or methyl, with compound of formula (III) as claimed in claim 16, 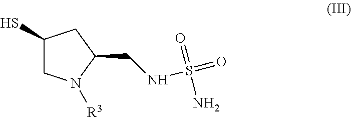
wherein R³ is p-nitrobenzyloxycarbonyl in the presence of anion exchange resin optionally in the presence of solvent;
ii) quenching the step (i) reaction mass in solvent(s);
iii) optionally isolating the compound of formula (IV); 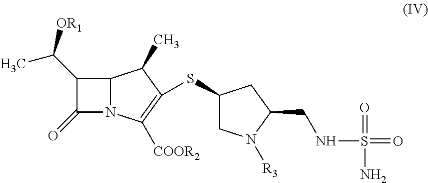
wherein R¹, R², R³ are as defined above;
iv) deprotecting the protecting group in a solvent, hydrogen pressure and in the presence of metal catalyst;
extracting Doripenem in water; and isolating the doripenem compound of formula (I) or its salts, hydrates and esters.
